(19) United States
(12) Patent Application Publication
(10) Pub. No.: US 2009/0048451 A1
(43) Pub. Date: Feb. 19, 2009

(54) Synthesis of triazole derivatives from Lewis base mediated nitroalkene-aldehyde coupling

(75) Inventor: Xiaodong Shi, Morgantown, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(73) Assignee: West Virginia University

(21) Appl. No.: 12/228,924

(22) Filed: Aug. 18, 2008

Related U.S. Application Data
(60) Provisional application No. 60/964,995, filed on Aug. 16, 2007.

(51) Int. Cl. C07D 249/04

(52) U.S. Cl. 548/255

(57) ABSTRACT

In the present invention a method to synthesize a triazole derivative from a Lewis base mediated nitroalkene-aldehyde coupling is revealed. In this synthesis a nitroalkene is reacted with an aromatic aldehyde in the presence of a Lewis Base, NaN₃, and a solvent for about 2 hours to about 48 hours at ambient conditions such as room temperature. The synthesized product may be any type of 4,5 disubstituted-1,2,3-triazoles. Of the nitroalkenes alkenes most β-alkyl nitroalkenes can react such as a β-substituted alkene. The aromatic aldehyde can be any 6 carbon aromatic aldehyde whether unsubstituted, substituted, disubstituted, heterocyclic, or a five carbon thiophene carboxaldehyde. The reaction takes place in the presence of NaN₃, a Lewis base, and solvents.

### CROSS-REFERENCE TO RELATED APPLICATIONS

This application claims priority to U.S. provisional application numbered 60/964,995.

### REFERENCE TO SEQUENCE LISTING, A TABLE, OR A COMPUTER PROGRAM LISTING COMPACT DISC APPENDIX

Not Applicable

### BACKGROUND OF THE INVENTION

Since the discovery of Cu-catalyzed azide-alkyne 1,3-dipolar cycloaddition (CuAAC, often referred as “click-chemistry”) early this century, the 1,2,3-triazole compounds have gained numerous attentions from scientists in various fields. Within the last five years, the importance of these compounds has been continuously demonstrated in the research fields as diverse as material science, chemical biology and medicinal chemistry. The classic synthesis of 1,2,3-triazole compounds was from the thermal reaction between azide and alkynes, which was reported more than a century ago. Rolf Huisgen and co-workers has done a comprehensive investigation for this reaction during 1950s to 1970s. In general, the thermal reaction suffering from the low overall reaction yields and limited substrate scopes, which resulted in few reported applications of this compounds before 2000. In 2002, Sharpless and co-workers reported the first copper (I) catalyzed 1,3-dipolar cycloaddition between the terminal alkyne and alkyl azide. This discovery provided a very efficient approach to the synthesis of diverse 1,4-disubstituted-1,2,3-triazoles compounds through “near-perfect” C—N bond formation reactions. Since then, applications of this strategy in other fields have been rapidly developed and many excellent works were reported. Meanwhile, the triazole compounds were also quickly identified as one important heterocyclic pharmacophore and were developed in to anti-cancer, anti-viral, and antibiotic agents.

As a very efficient method for the synthesis of N-substituted triazole, the CuAAC did not work very well with non-substituted azide, such as NaN3 and HN3. Therefore, to prepare the N-unsubstituted triazoles (NH-triazole) by CuAAC, the organic azides are usually applied with removable N-protecting groups. Meanwhile, the thermal reactions between alkyne and NaN3/HN3 gave low yields and the preparation of substituted alkynes was usually high cost. With the continuous interest and need of the triazole derivatives, the efficient synthesis of highly substituted NH-triazoles (as an complementary approach for the CuAAC reaction) is highly desirable.

### BRIEF SUMMARY OF THE INVENTION

One embodiment is a method to synthesize triazole derivative from a Lewis base mediated nitroalkene-aldehyde-azide coupling is revealed. In this synthesis a nitroalkene is reacted with an aromatic aldehyde in the presence of proline, NaN₃, and a solvent for about 2 to about 48 hours at ambient conditions such as room temperature. The synthesized products may be any type of 4,5 disubstituted-1,2,3-triazoles.

An aspect of the embodiment is the use of a nitroalkene as a reactant. Of the nitroalkenes most β-alkyl nitroalkenes can react such as a β-substituted nitrostyrene.

A further aspect of the embodiment is the aromatic aldehyde as a reactant where the aromatic aldehyde may be any 6 carbon unsubstituted, substituted, disubstituted, trisubstituted, tetrasubstituted, pentasubstituted, heterocyclic, or a five carbon heterocyclic carboxaldehyde.

Another aspect of the embodiment is that the reaction takes place in the presence of NaN₃, any secondary amine, and solvents.

### BRIEF DESCRIPTION OF THE SEVERAL VEIWS OF THE DRAWING

Not Available

### DETAILED DESCRIPTION OF THE INVENTION

The present invention details a method to synthesize a triazole derivative from a Lewis base mediated nitroalkene-aldehyde coupling. In this synthesis a nitroalkene is reacted with an aromatic aldehyde in the presence of a secondary amine such as NaN₃, di-alkyl, alkyl-aryl and aryl-aryl secondary amines, and a solvent for about 2 to about 48 hours at ambient conditions ranging from about room temperature to about 150° C. The synthesized product may be any type of 4,5 disubstituted-1,2,3-triazoles where Ph is a phenyl and Ar is an aromatic group. An example of the synthesis is:

### EXAMPLE 1

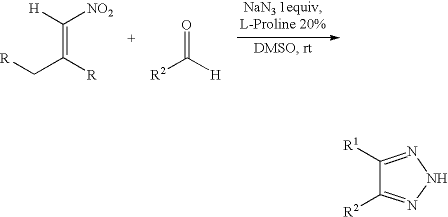

The first reactant of the synthetic equation of Example 1 is the nitroalkene. This method is suitable for all nitroalkenes. Polymerization of the nitroalkene side product is possible during the reaction which makes nitroalkenes a good working reactant. Any β-substituted alkenes are suitable and can be:

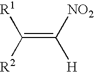

where R¹═H, CH₃, or an alkyl chain and R²=any alkyl group or aryl group. Many nitroalkenes have already been attempted in the reactions with success. Some successful nitroalkenes are:

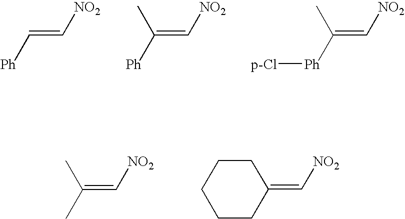

The second reactant in the chemical equation is the aromatic aldehyde. The aromatic aldehyde may be any 6 ring carbon whether unsubstituted, substituted, disubstituted, trisubstituted, tetrasubstituted, pentasubstituted, heterocyclic, or a five carbon heterocyclic carboxaldehyde. Testing of unsubstituted aromatic aldehydes resulted in benzaldehyde and napthaladehyde both successfully reacting during the synthesis. Substituted aldehydes with both electron donating and electron withdrawing groups are able to react. A substituted aromatic aldehyde can be successful with any of the following:

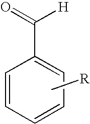

where R can be at any otho, para, or meta substitution and can be NO₂, CN, Cl, Br, OH, OMe where Me is any methyl group, or any alkyl group. Disubstituted aromatic aldehydes also react in the synthetic equation such as:

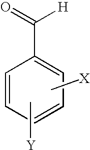

where X and Y can be NO₂, CN, Cl, Br, OH, OMe where Me is any methyl group, or any alkyl group and X and Y may occupy any possible substitution position such as 2,4; 2,5; 2,6; 3,5 etc. such as:

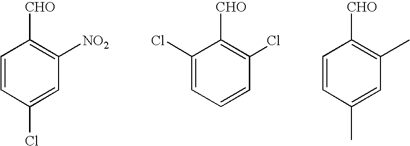

Among the aromatic aldehydes heterocyclic aromatic aldehydes may also be used. Heterocyclic aromatic aldehydes such as pyridine carboxaldehyde yield excellent results. A pyridine carboxaldehyde can be:

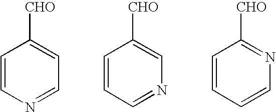

Of five member heterocyclic aldehydes carboxaldehyde has been the most successful:

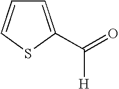

The reaction takes place in the presence of NaN₃, Lewis base, and solvents. NaN₃ is used in the amounts of about 1 to 4 equivalent, where they all gave desired products with various of different yields slightly. The Lewis base can be any secondary amine such as L-Proline or prolinol and about 5 to 100% is added to the reactants. A wide variety of solvents may be used such as alcohols, water, DMF, acetone, acetonitrile, tetrahydrofuran, or DMSO between 0.1 M to 1 M may be used with the concentration of nitroalkenes.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method to make any 4,5 disubstituted-1,2,3-triazole comprising an effective amount of a ntiroalkene reacted with an effective amount of an aromatic aldehyde in the presence of an effective amount of a secondary amine, and an effective amount of a solvent for an effective time at effective conditions.

2. The method to make any 4,5 disubstituted-1,2,3-triazole of claim 1 wherein the nitroalkene is 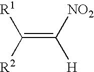
where R¹ is H, CH₃, or an alkyl chain and R² is any alkyl group or aryl group.

3. The method to make any 4,5 disubstituted-1,2,3-triazole of claim 1 wherein the aromatic aldehyde is the substituted aromatic aldehyde 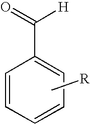
where R is either an otho, para, or meta substitution and R is one of NO₂, CN, Cl, Br, OH, OMe where Me is any methyl group, or any alkyl group.

4. The method to make any 4,5 disubstituted-1,2,3-triazole of claim 1 wherein the aromatic aldehyde is the disubstituted aromatic aldehyde 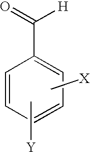
where X and Y are one or more of NO₂, CN, Cl, Br, OH, OMe where Me is any methyl group, or any alkyl group and X and Y may occupy any possible substitution position.

5. The method to make any 4,5 disubstituted-1,2,3-triazole of claim 1 wherein the aromatic aldehyde is a heterocyclic aromatic aldehyde.

6. The method to make any 4,5 disubstituted-1,2,3-triazole of claim 5 wherein the heterocyclic aromatic aldehyde is a pyridine carboxaldehyde.

7. The method to make any 4,5 disubstituted-1,2,3-triazole of claim 1 wherein the aromatic aldehyde is a five carbon heterocyclic carboxaldehyde.

8. The method to make any 4,5 disubstituted-1,2,3-triazole of claim 1 wherein the secondary amine is NaN₃.

9. The method to make any 4,5 disubstituted-1,2,3-triazole of claim 1 wherein said solvent is one or more of the solvents alcohol, water, DMF, acetone, acetonitrile, tetrahydrofuran, and DMSO.

10. A method to synthesize a triazole derivative from a Lewis base mediated nitroalkene-aldehyde coupling comprising reacting an effective amount of a nitroalkene with an effective amount of an aromatic aldehyde in the presence of an effective amount of a Lewis Base and an effective amount of a solvent for an effective time at effective conditions.

11. The method to make any 4,5 disubstituted-1,2,3-triazole of claim 10 wherein the nitroalkene is 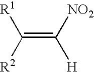
where R¹ is H, CH₃, or an alkyl chain and R² is any alkyl group or aryl group.

12. The method to make any 4,5 disubstituted-1,2,3-triazole of claim 10 wherein the aromatic aldehyde is the substituted aromatic aldehyde 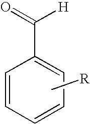
where R is either an otho, para, or meta substitution and R is one of NO₂, CN, Cl, Br, OH, OMe where Me is any methyl group, or any alkyl group.

13. The method to make any 4,5 disubstituted-1,2,3-triazole of claim 10 wherein the aromatic aldehyde is the disubstituted aromatic aldehyde 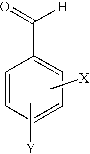
where X and Y are one or more of NO₂, CN, Cl, Br, OH, OMe where Me is any methyl group, or any alkyl group and X and Y may occupy any possible substitution position.

14. The method to make any 4,5 disubstituted-1,2,3-triazole of claim 10 wherein the aromatic aldehyde is a heterocyclic aromatic aldehyde.

15. The method to make any 4,5 disubstituted-1,2,3-triazole of claim 14 wherein the heterocyclic aromatic aldehyde is a pyridine carboxaldehyde.

16. The method to make any 4,5 disubstituted-1,2,3-triazole of claim 10 wherein the aromatic aldehyde is a five carbon heterocyclic carboxaldehyde.

17. The method to make any 4,5 disubstituted-1,2,3-triazole of claim 10 wherein the Lewis Base is NaN₃.

18. The method to make any 4,5 disubstituted-1,2,3-triazole of claim 10 wherein said solvent is one or more of the solvents alcohol, water, DMF, acetone, acetonitrile, tetrahydrofuran, and DMSO.
